(19) United States
(12) Patent Application Publication
(10) Pub. No.: US 2008/0187581 A1
(43) Pub. Date: Aug. 7, 2008

(54) Delivery System For Mulitple Drugs

(76) Inventors: Subhash Pandurang Gore, Hyderabad (IN); Verma Sanjay, Secunderabad (IN); Vijay Dinanath Nasare, Navi Mumbai (IN); Indu Bhushan, Hyderabad (IN); Venkata Nookaraju Sreedharala, Hyderabad (IN); Harshal Prabhakar Bhagwatwar, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/908,631

(22) PCT Filed: Mar. 16, 2006
(86) PCT No.: PCT/US06/10036

§ 371 Date: Sep. 14, 2007

(30) Foreign Application Priority Data
Mar. 16, 2005 (IN) 267/CHE/2005
Dec. 20, 2005 (IN) 1862/CHE/2005

(51) Int. Cl. A61K 9/48; A61K 31/135; A61P 43/00

(52) U.S. Cl. 424/463; 514/658

(57) ABSTRACT

A solid pharmaceutical dosage form comprises a substrate containing an active agent and a solid composition deposited onto areas of a surface of the substrate and containing an active agent.

### INTRODUCTION TO THE INVENTION

The present invention relates to drug delivery systems for the simultaneous delivery of multiple drug substances. In an aspect, the invention relates to the stabilization and simultaneous delivery of incompatible drug substances or drug substances requiring different delivery profiles from the same composition.

Unit dose compositions of incompatible drugs or of a combination of immediate release active agent(s) or optionally with a modified release active agent have been described in the literature. Various techniques and methods known for such simultaneous delivery include coating one of the actives with an inert material to reduce the contact with the other drug substance (U.S. Pat. No. 5,593,696 describes coating of famotidine with an inert material to combine it with sucralfate in a unit dosage form, which otherwise is not stable), or to modify the release characteristics of the active (U.S. Pat. No. 6,039,974 describes a pharmaceutical composition for the combination of a therapeutically effective decongestant as a sustained release layer and antihistaminic amount of piperidinoalkanol such as loratidine, desloratidine and the like as immediate release layer); a tablet formulation containing immediate release and delayed release granules (U.S. Pat. Nos. 6,183,779 and 6,287,600 describe a solid pharmaceutical composition comprising a therapeutically effective amount of enterically coated granules of a nonsteroidal anti inflammatory drug (NSAID), an effective anti-ulcerogenic amount of a prostaglandin and a prostaglandin stabilizer); a two or three layer tablet (U.S. Pat. No. 6,087,386 describes use of a bilayer tablet principle for a pharmaceutical composition of enalapril and losartan, wherein each active is present in different layers of the tablet); a tablet with a delayed release core surrounded by an immediate release shell, a tablet with two or more actives, wherein one active is in the form of coated pellets (U.S. Pat. No. 6,511,680 describes a tablet containing a NSAID and misoprostol, wherein NSAID is in the form of coated pellets); a delayed release tablet/granule coated with a film of an immediate release active agent, an inlay tablet (WO 2004/012700), a capsule containing pellets to name a few of them.

U.S. Application Publication No. 2005/0163847 describes a solid oral dosage form comprising a first portion of a NSAID and a coating comprising an antiulcerative compound. The said coating at least partially surrounds the first NSAID portion. Also it discloses a solid oral dosage form comprising a NSAID portion having at least one internal hole extending through said portion and a coating portion comprising an antiulcerative compound at least partially filling the internal hole of the NSAID portion.

The selection of a suitable dosage form and composition for the stabilization and simultaneous delivery of incompatible active agents poses a particularly strong challenge to the pharmaceutical formulations scientist. Such simultaneous delivery depends on variables like the physicochemical properties of the active agents, mode of degradation of the actives, known interactions of the actives and also with the excipients, the dose to be administered and the intended site of action to name a few. Also, the combination of active agents is difficult to formulate due to the inherent differences in physicochemical properties, drug-drug, drug-excipient incompatibilities, and the type of release profiles needed for each of the active agents to elicit the necessary therapeutic efficacy.

The combined administration of actives is expected to result in a beneficial and potentiating or synergistic therapeutic effect when compared with the administration of the two actives alone. Moreover, the possible therapeutic benefits resulting from the use of a combination for treatment are prolongation of efficacy, a broad therapeutic treatment of diseases and conditions, enhanced patient compliance because of a reduced dosing frequency, among other advantages.

For a combination therapy, the active agents should be compatible with one another as well as the excipients used in the composition so as to form a stable and efficacious formulation in order to maximize the therapeutic activity. Earlier introduction of stable and efficacious formulations for a combination therapy is increasingly being recommended.

There is a continuing need for the development of alternative pharmaceutical compositions for the simultaneous delivery of incompatible active substances from the same unit dose composition.

These and other needs are addressed by this invention.

### SUMMARY OF THE INVENTION

The present invention relates to drug delivery systems for the simultaneous delivery of multiple drug substances.

More specifically, the invention relates to the simultaneous delivery of incompatible drug substances or drug substances requiring different delivery profiles, from the same composition.

Such drug delivery systems are provided in the form of solid compositions comprising:
a) a substrate-platform comprising one or more active agents and wherein another active agent is applied as a band over the substrate-platform; or
b) a substrate-platform comprising one or more active agents and wherein another active agent is incorporated in ink used for embossing or printing on the substrate-platform; or
c) tablets comprising at least two layers wherein at least one of the layers contains at least one indentation wherein the said indentation in the tablets contains added therein at least one composition comprising a first active agent and the other layer comprises a second active agent.

In an embodiment, a solid pharmaceutical dosage form comprises a substrate containing an active agent and a solid composition deposited onto areas of a surface of the substrate and containing an active agent.

In another embodiment, a solid pharmaceutical dosage form comprises a substrate containing an active agent and a solid composition deposited into one or more recessed areas on at least one surface of the substrate and containing a different active agent.

In a further embodiment, a solid pharmaceutical dosage form comprises a substrate containing an active agent and a solid composition deposited as a band onto at least one surface of the substrate and containing a different active agent.

### BRIEF DESCRIPTION OF THE DRAWINGS

FIG. 1 is a side view of a tablet applied with a band about the circumference according to Example 1.

FIG. 2 is a side view of a capsule applied with a band near the capsule cap and body joint according to Example 2.

FIG. 3 is a top view of a tablet, wherein one of the active ingredients is incorporated in ink used for imprinting according to Example 6.

FIG. 4 is a top view of a bilayer caplet-shaped tablet with two indentations.

FIG. 5 is a side view of a bilayer tablet.

FIG. 6 is a top view of a bilayer triangular tablet.

FIG. 7 is a top view of a bilayer round tablet having three round-shaped indentations on one surface.

FIG. 8a is top view of a trilayer caplet-shaped tablet having two indentations, each of different size and shape.

FIG. 9 is a side view of a bilayer tablet having a flat surface and another surface concave.

### DETAILED DESCRIPTION OF THE INVENTION

The present invention, in an aspect, relates to drug delivery systems for the simultaneous delivery of multiple drug substances wherein the drug substances are chemically incompatible with one another or require different delivery profiles from the same composition. The invention includes solid dosage forms having a “substrate” that contains at least one drug substance and physically comprises a larger portion of the dosage form, the substrate having an applied band, layer, imprint, or other surface application of a composition that contains one or more other drug substances. The composition will be applied to specific areas of the substrate, but will not completely or substantially completely coat the substrate.

In another aspect, the invention relates to solid dosage forms having the same drug substance in more than one composition, where each composition releases the drug at a different rate. For example a tablet body can provide delayed or controlled release of the drug, while an applied band, layer, imprint, etc. can provide an immediate release profile or a delayed or controlled release that is different from the release profile from the body.

The terms “drug substance,” “active agent,” and “active” are used interchangeably herein and refer to the pharmacologically active components of the pharmaceutical dosage forms of the invention.

Specific embodiments of such drug delivery systems are provided in the form of solid compositions comprising:
a) a substrate comprising one or more active agents and wherein the other active agent is applied as a band over the substrate; or
b) a substrate comprising one or more active agents wherein another active agent is incorporated in ink used for printing on the substrate; or
c) tablets comprising at least two layers wherein at least one of the layers at least one composition comprising a first active agent, containing at least one indentation that contains a composition comprising a second active agent.
   In any of these embodiments, a single active agent can be used for any of the substrate and the band, ink, or filling of an indentation.

The term “substrate” used herein represents a solid platform or body, upon which another active agent can be deposited; examples include, but are not limited to, tablets and hard and soft gelatin capsules.

The term “band”, is defined as the physical deposit of at least one active agent composition about the circumference of tablet or capsule either completely or partially. Banding can be partial with respect to horizontal or vertical or both the axes over the circumference of tablet or capsule.

“Text or logo” is defined as any written matter on a tablet or capsule surface including alphabets, numericals, symbols, characters, drawings, designs, lines, pictures or figures.

“Ink” as used herein refers to an imprinting composition with or without colors or dyes.

“Deposit” is defined as the deposit of an active agent-containing composition on the indentation, at least partially or completely filling the indentation such that the active agent composition provides an immediate or modified release of the active agent, or both.

The term “indentation” refers to any depression or recessed area on the surface of at least one of the layers of a tablet, which can hold an active agent-delivering composition. Indentations could comprise scoring lines or breakline(s), embossing(s), internal holes, cavities, hollow cores, concavity, recesses; donut-shaped configurations and notches that do not extend throughout the said layer. These terms and others may be used synonymously for “indentation.”

Stability of pharmaceutical compositions may be defined as the capability of a particular dosage form, in its packaging, to maintain its physical, chemical, microbiological, therapeutic and toxicological specifications during a period of storage and use. Stability of pharmaceutical compositions may be affected by several factors, including the stability of the drug substance, drug substance-drug substance interaction and drug substance-excipient incompatibility. The present invention relates to the delivery systems in the form of solid compositions such as for example tablets or capsules. Tablets useful in the invention are of shapes known to the person skilled in the art, such as for example but not limited to round, caplet, oblong, oval, elliptical, triangular, square, rectangular, hexagonal, heptagonal, polygonal and the like. The edges of the tablets may be rounded, pointed, blunt and the like. Tablet surfaces may be flat, flat face beveled edge, normal concave, deep concave or any concavity that is known to a person skilled in the art to suit the tablet size and shape to aid the processing of the formulation during compression, coating and packing. Capsules useful in the invention are usually of hard gelatin or soft gelatin and are of shapes known to the person skilled in the art, such as for example round, oblong, oval, elliptical and the like.

In an embodiment of the invention, an uncoated or coated tablet or capsule comprising one or more active agents with or without pharmaceutically acceptable excipients is deposited with another active agent with or without pharmaceutically acceptable excipients as one or more bands, particularly across the circumference of the tablet or near the capsule cap-body joint, partially or completely.

The active agent is dissolved or dispersed with or without a binder and optionally with other pharmaceutically acceptable excipients in a suitable solvent, and is applied as a band over the circumference of the tablet or capsule comprising other active agents. Binders that may be used in the present invention include but are not limited to: gelatin; gums such as agarose, gum arabic, gum ghatti, gum karaya, gum tragacanth; hydrophilic colloids such as alginates; other substances such as arbinoglactan, pectin, amylopectin, N-vinyl lactams, polysaccharides and the like; celluloses such as sodium carboxymethyl cellulose, ethyl cellulose, hydroxylpropyl cellulose, hydroxylpropyl methyl cellulose; polyvinyl pyrrolidone; and starches and modified starches to name a few. Other binders or mixtures of two or more binders may be used in the preparation of the bands as required and are all within the scope of the invention. Examples of solvents that are used include without limitation water, isopropyl alcohol, ethanol, dichloromethane, acetone, or combinations thereof. Other pharmaceutically acceptable excipients may include plasticizers, wetting agents, surfactants, colorants, flavors, taste-masking agents and the like.

Capsule banding machines may be used to deposit the band over the tablet or capsule or other techniques such as spraying, dip coating, rolling and the like also may be used.

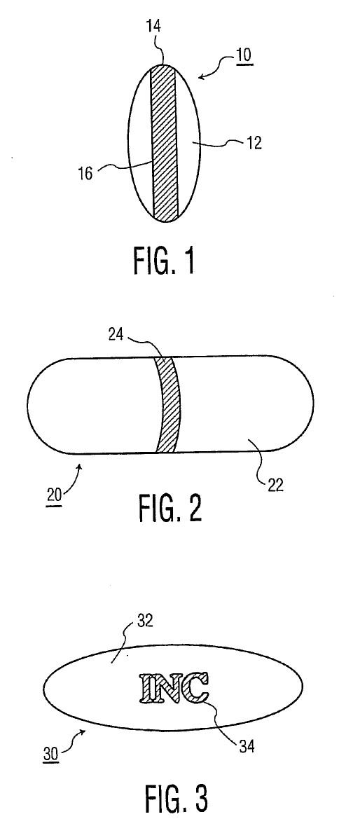

FIG. 1 shows a side view of a tablet according to one embodiment of the invention. Capsule-shaped tablet 10 has a surface 12 and circumference 14 deposited with a band 16. The band can extend over any desired length and width of the circumference. It can also be applied as single band or as multiple bands which are either linear or non-linear, although it is shown as being a single band extended over the full length and of the maximum circumference.

FIG. 2 shows a side view of a capsule according to an embodiment of the invention. Capsule 20 has a surface 22 deposited with a band 24. The band can extend over any desired length and width of the surface. It can be deposited at any position over the surface of the capsule and also can be applied as single band or multiple bands which are either linear or non-linear, although it is shown as being a single band extending over the full diameter, covering the capsule cap-body joint.

In another embodiment of the invention, one or more of the active agents is dissolved or dispersed in an ink material comprising pharmaceutically acceptable colors or dyes or lakes, and the solution or dispersion is used to write text or draw logos or pictures or figures or symbols over a tablet or capsule comprising other active agents with or without pharmaceutically acceptable excipients.

FIG. 3 shows a top view of a tablet according to an embodiment of the invention. Tablet 30 has an upper surface 32 printed with text 34. Printing can be straight, circular, or in any other configuration on any surface as letters, numbers, symbols, characters, drawings, designs, logos, lines, pictures or figures, either visible or non-visible, although it is shown as being text which is visible on upper surface.

Indentations that are part of tablets as per the present invention can comprise one or multiple indentations. Different shapes, sizes and positions on the tablet characterize the said indentations. The shape of an indentation can be round, oval, elliptical, square, rectangular, triangular, hexagonal, heptagonal, polygonal and the like. Size or dimensions of indentations such as length, width, radius and depth may or may not be proportional to the size or shape of the tablet or amount of active pharmaceutical ingredient proposed to be deposited into it, or the number of indentations present on the tablet. Indentations are positioned on the tablets either lengthwise, width-wise, centrally or to side(s) or corner(s). Also, the same or different shapes or sizes of indentations may be placed on a single tablet. Indentation(s) on the tablets may be designed either on upper or lower surfaces, or both. Equal or unequal numbers of indentations on the upper and lower surfaces of the tablet are also within the scope of the invention.

In one embodiment of the present invention, an indentation that is a part of the tablet is formed on one side of the tablet surface by providing a convex projection of the tablet tooling.

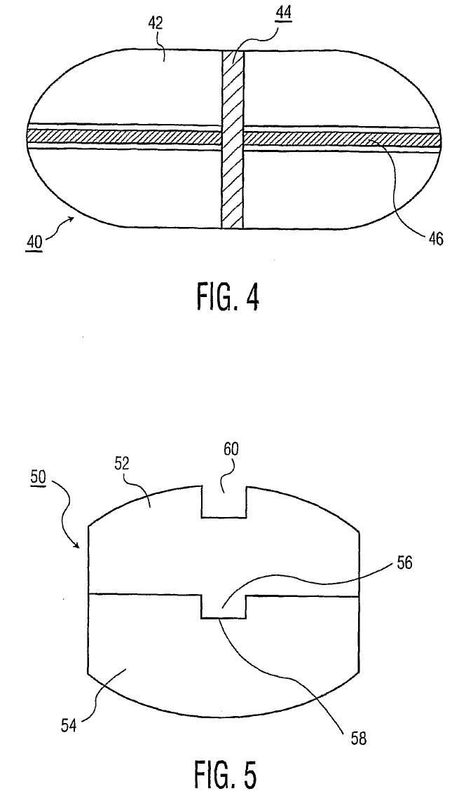

FIG. 4 shows a top view of a tablet, according to one embodiment of the invention. Tablet 40 has a surface 42 provided with a longitudinal indentation or groove 44 that extends into the surface. Indentation 44 can extend over any desired portion of the tablet length or width and will not necessarily extend over an entire width or length, as is shown in the drawing. Indentation 44 can be located at any desired portion of the surface, although it is shown as being centered in the length of the tablet. Optionally, a transverse indentation or groove 46 can also be provided, extending into the surface. Indentation 46 can extend over any desired portion of the tablet width, and will not necessarily extend over the entire width, as is shown in the drawing. Further, indentation 46 can be located at any desired portion of the surface, although it is shown as being centered in the length of the tablet. Indentations 44 and 46 may or may not intersect, and may intersect at an oblique angle.

In FIG. 5, tablet 50 is shown in a cross-sectional end view, comprising upper layer 52 and lower layer 54. Upper layer 52 is provided with a projection 56 that is positioned within recess 58 of lower layer 54, assisting in maintaining a stable layered structure of the tablet. Recess 58 can be formed by a projecting area on a tablet machine punch that is used to compress lower layer 54, and then projection 56 will be directly formed within recess 58 when upper layer 52 is formed by subsequently compressing material onto lower layer 54. Indentation 60 extends into a portion of upper layer 52, to a desired depth that is less than the thickness of the layer. Upper layer 52 can also optionally be provided with a recess or indentation similar to indentation 60, although this feature is not shown.

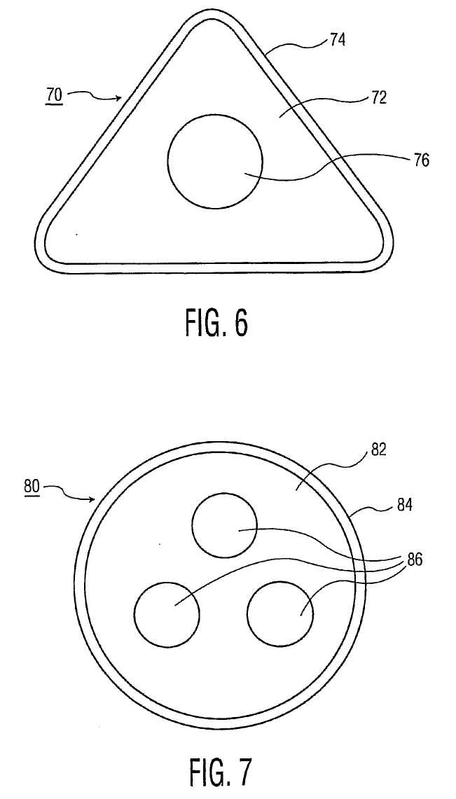

In FIG. 6, triangular tablet 70 is shown in a top view, comprising upper layer 72 and a slightly larger lower layer 74 that is in the background of the drawing. The layers can conveniently be made the same sizes and shapes, the drawing representing the lower layer as somewhat larger for purposes of illustration. Upper layer 72 is provided with an indentation 76, which is shown as being circular. Indentation 76 can be of any desired size, depth and shape, and can be positioned at any location on the tablet upper or lower layer, although it is shown for purposes of illustration as a circular indentation positioned at the center of the tablet upper layer.

In FIG. 7, bilayer tablet 80 is shown in a top view, comprising upper layer 82 and lower layer 84 that is in the background of the drawing and shown as being slightly larger for clarity. Typically, the layers would have approximately the same diameters. Upper layer 82 is provided with three indentations 86 which are shown as being circular. Indentations 86 can be of any number, size, depth and shape, and can be positioned at any locations on the tablet upper or lower layer, although they is shown as circular indentations positioned on the tablet upper layer.

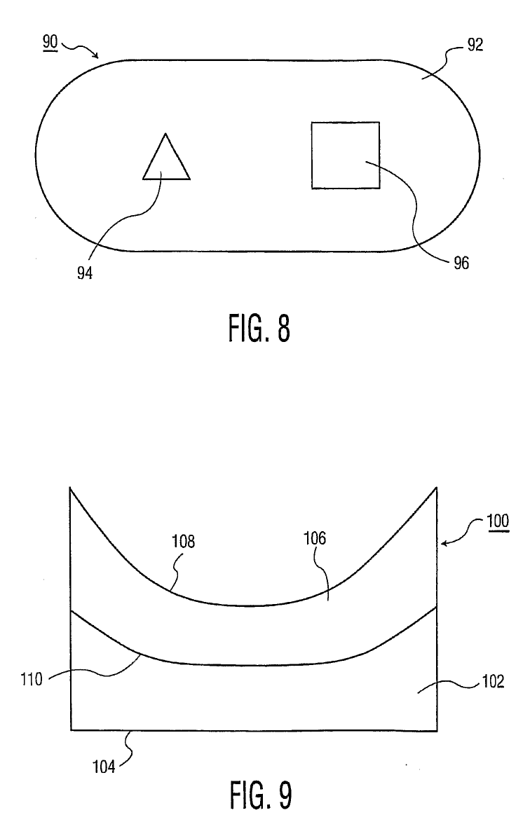

In FIG. 8, tablet 90 is shown in a top view, comprising upper surface 92. Upper surface 92 is provided with two indentations, one triangular 94 and the other square 96. Indentations can be of any number, size, depth and shape, and can be positioned at any location on the tablet upper or lower surface, although they are shown as including triangular and square indentations positioned on the tablet upper surface.

FIG. 9 shows a cross-sectional view of bilayer tablet 100 according to one embodiment, having lower layer 102 with a lower flat surface 104 and upper layer 106 with an upper surface 108 that is concave. The interface 110 between the layers is depicted as having the same contour as upper surface 108, but can be made essentially flat or have any other desired contour.

In an embodiment, the compositions of the present invention can be made as layered tablets comprising at least two layers wherein one layer comprises an active agent (A) and other layer forms either an inert layer or a layer comprising another active agent (B) and a deposit of yet another active agent (C) which is incompatible with active (A) is present in indentation(s) such that the indentation remains within the said inert or active agent (B) layer.

In another embodiment, the drug delivery system comprises a bilayer tablet, wherein one layer comprises active agent (A), the second layer comprises an inert layer or a layer of another active agent (B) and the second layer comprises more than two indentations that remain within the second layer such that each indent is deposited with different active agents which are incompatible with each other and with active agent (A).

In yet another embodiment, a layered tablet includes two or more layers, wherein more than one layer comprises an active agent (A), but individual layers are formulated such as to release the active agent (A) at different rates. The tablet can also have one or more layers that contain a different active agent (B), and further optionally can be provided with one or more indentations on at least one surface, containing an active agent that would be incompatible with the active agent in any layer of the tablet.

In still another embodiment, an indentation is at least partially or completely filled with a deposit comprising active agent(s) in an immediate release or modified release formulation or both.

Further, there may be more than one score line or breakline on at least one of the layers of a bi or tri-layered tablet. The breaklines may be either parallel to one another or intersect one another or in any other manner known to a person skilled in the art.

In another embodiment, different kinds of embossings as are known in the art fall within the scope of the invention with respect to shape, size and dimension of the same.

In yet another embodiment, the layered tablets can be in the shape of a circle, triangle, square, rectangle, hexagon and the like.

In one embodiment of the invention there is provided a composition for the simultaneous delivery of two or more incompatible drug substances in a stable unit dose composition.

In another embodiment, a unit dose composition of more than two actives is provided wherein the actives have different release profiles.

In yet another embodiment, a unit dose composition for simultaneous delivery of drug substances is provided wherein one of the actives is incompatible with any of the excipients used for the delivery of another active in the same composition.

In a further embodiment of the invention, a unit dose composition for simultaneous delivery of drug substances is provided wherein the release of one of the actives would be undesirably modified because of excipients used for the delivery of another active from the same composition.

The present invention thus provides stable compositions for reducing drug-drug or drug-excipient interactions associated with the simultaneous administration of more than one active agent from the same composition or provides incompatible active agents in an immediate release or modified release formulation or both concomitantly from a single pharmaceutical composition.

Various active agents or their pharmaceutically acceptable salts, solvates, enantiomers or a mixture thereof known to a person skilled in the art falls within the scope of this invention. Representative active agents include but are not limited to: non-steroidal anti-inflammatory drugs (NSAIDs) such as propionic acid derivatives like ibuprofen, naproxen, flurbiprofen, fenoprofen, ketoprofen, suprofen, fenbufen or fluprofen; acetic acid derivatives like tolmetin sodium, zomepirac, sulindac or indomethacin; fenamic acid derivatives like mefenamic acid or meclofenamate sodium; biphenylcarboxylic acid derivatives like diflunisal or flufenisal; oxicams like piroxicam, sudoxicam or isoxicam; benzeneacetic acid derivatives like diclofenac; COX-2 inhibitors like celecoxib or rofecoxib; ulcer protective prostaglandins or their analogues such as misoprostol, carboprost, ornoprostil, dinoprost, gemeprost, metenoprost, sulprostone or tiaprost; bronchodilators like guaiphenesin; anti-tussitives such as dextromethorphan, codeine or pholcodine; opoid analgesics like naproxen or naltrexone, HMG CoA reductase inhibitors such as atorvastatin, cerivastatin, fluvastatin, lovastatin, pravastatin, rosuvastatin, simvastatin, pitvastatin, fluindostatin, mevastatin, velostatin or dalvastatin; biguanides such as metformin, phenformin or buformin; sulfonylureas such as acetohexamide, chlorpropamide, glimepiride, glipizide, glyburide, tolazamide, tolbutamide, gliclazide, glibornuride, glisoxepide, tolazamide, phenbulamide or tolcyclamide; other classes of antidiabetic agents including: glitazones such as rosiglitazone, troglitazone or pioglitazone; glucosidase inhibitors such as acarbose or meglitol; meglitinides such as nateglinide, repaglinide; lipid lowering agents such as fibrates including clofibrate, gemfibrozil or fenofibrate and the like.

It is to be understood that the invention is not limited to the actives mentioned above and any other active agents may also be delivered using these compositions.

In yet another embodiment, an aqueous or nonaqueous enteric coating can optionally be used to aid in controlling the release of the active. The enteric coating aids in the prevention of the acid labile active with the acidic gastric juices after oral administration as well as providing direct delivery of the active in the lower gastrointestinal tract rather than in the stomach. Suitable enteric-coating polymers include but are not limited to the different grades of anionic polymers of methacrylic acid and methacrylates, such as but not limited to Eudragit L100-55™, spray dried Eudragit L30D-55 ™, Eudragit L30D-55 ™, Eudragit L100 ™, Eudragit S100™ and Eudragit FS30D™.

Release of active agent from compositions of the present invention can be modified by using rate controlling agents including, but not limited to: water soluble polymers of various grades such as celluloses such as methylcellulose, carboxymethyl cellulose, hydroxypropyl methylcellulose, cross-linked sodium carboxymethyl cellulose and cross-linked hydroxypropyl cellulose; carboxymethylamide; potassium methacrylate/divinylbenzene copolymer; polymethylmethacrylate; polyhydroxyalkyl methacrylate; cross-linked polyvinylpyrrolidone; gums such as agarose, gum arabic, gum ghatti, gum karaya, gum tragacanth; hydrophilic colloids such as alginates; other substances such as arbinoglactan, pectin, amylopectin, N-vinyl lactams, polysaccharides and the like.

Water-insoluble polymers or combinations thereof used in various ratios for coating compositions are exemplified by, but are not limited to: oils; waxes such as beeswax, carnauba wax, and microcrystalline wax; fatty alcohols such as cetostearyl alcohol, stearyl alcohol, cetyl alcohol, and myristyl alcohol; fatty acid esters such as glyceryl monostearate, glycerol distearate, glycerol monooleate, acetylated monoglycerides, tristearin, tripalmitin, cetyl esters wax, glyceryl palmitostearate, and glyceryl behenate; celluloses such as ethyl cellulose, low substituted hydroxylpropyl cellulose (L-HPC), cellulose acetates, and their derivatives, cellulose acetate phthalate, hydroxylpropyl methylcellulose phthalate, cellulose acylate, cellulose diacylate, cellulose triacylate, cellulose acetate, cellulose diacetate, cellulose triacetate, mono-, di- and tri-cellulose alkanylates, mono-, di-, and tri-cellulose arylates, and mono-, di- and tri-cellulose alkenylates; polymers, polymethacrylic acid based polymers and copolymers sold using the trademark EUDRAGIT (Eudragit RL and RS, NE-30D); zein; aliphatic polyesters; copolymers of the above polymers; or mixtures of any two or more in various ratios and proportions as required are within the scope of this invention without limitation. Of course, any other polymer, which aids in modulated release, is also acceptable in the practice of this invention. These agents alone or in combination with different classes of rate controlling substances are used to control the release of the active substances by matrix or reservoir of combination principles.

Layered tablets containing actives will also include usual tabletting excipients.

The pharmaceutical compositions of the present invention may contain one or more diluents to make up the tablet mass so that it becomes easier for the patient and the caregiver to handle. Common diluents that can be used in pharmaceutical formulations include microcrystalline cellulose (MCC), silicified MCC (e.g. Prosolv™ HD 90), micro fine cellulose, lactose, starch, pregelatinized starch, calcium carbonate, calcium sulfate, sugar, mannitol, sorbitol, dextrates, dextrin, maltodextrin, dextrose, dibasic calcium phosphate dehydrate, tribasic calcium phosphate, magnesium carbonate, magnesium oxide, and the like.

The pharmaceutical compositions to be made into tablets may further include a disintegrant. Disintegrants include but are not limited to methyl cellulose, microcrystalline cellulose, carboxymethyl cellulose calcium, carboxymethyl cellulose sodium (e.g. Ac-Di-Solo, Primellose®), crospovidone (e.g. Kollidon®, Polyplasdone®), povidone K-30, guar gum, magnesium aluminum silicate, colloidal silicon dioxide (Aerosil®), polacrilin potassium, starch, pregelatinized starch, sodium starch glycolate (e.g. Explotab®) and sodium alginate.

Any coating technique such as pan coating, fluid bed coating and the like known to a person skilled in the art falls within the scope of the present invention.

Useful plasticizers for coating are materials such as acetyl alkyl citrates, phosphate esters, phthalate esters, amides, mineral oils, fatty acids and esters thereof with polyethylene glycol, glycerin, triacetin or sugars, fatty alcohols, ethers of polyethylene glycol and vegetable oils. Useful fatty alcohols include cetostearyl alcohol, cetyl alcohol, stearyl alcohol, oleyl alcohol and myristyl alcohol.

Pharmaceutical compositions for tableting and film formation may further include but are not limited to pharmaceutically acceptable glidants, lubricants, flavoring agents, opacifiers, colorants and other commonly used excipients.

In an embodiment, the pharmaceutical compositions of the present invention are manufactured as described below. Granules containing each of the immediate release or controlled release or delayed release or sustained release actives are prepared separately by sifting the actives and excipients through the desired mesh size sieve and then are mixed using a rapid mixer granulator, planetary mixer, mass mixer, ribbon mixer, fluid bed processor or any other suitable device. The blend can be granulated, such as by adding a solvent like water, isopropyl alcohol, acetone, ethanol, dichloromethane, and the like and combinations thereof, or by adding a solution of a binder in any solvents described above in a low or high shear mixer, fluidized bed granulator and the like or by dry granulation. The granulate can be dried using a tray drier, fluid bed drier, rotary cone vacuum drier and the like. The sizing of the granules can be done using an oscillating granulator, comminuting mill or any other conventional equipment equipped with a suitable screen. Alternatively, granules can be prepared by extrusion and spheronization or roller compaction. The dried granulate particles are sieved, and then mixed with lubricants and disintegrants and compressed into a tablet, optionally comprising one or more indentations not extending through the said tablet.

Alternatively the manufacture of granules of the immediate release or controlled release or delayed release or sustained release actives can be made by mixing the directly compressible excipients or by roller compaction. The blend so obtained is compressed using a suitable device, such as a multi-station rotary machine to form slugs, which are passed through a multimill, fluid energy mill, ball mill, colloid mill, roller mill, hammer mill, and the like, equipped with a suitable screen. The milled slugs of the immediate release or controlled release or delayed release or sustained release actives are then lubricated and compressed into a tablet optionally comprising one or more indentations not extending through the tablet.

The deposit of the active on indentation(s) may either be done by compression coating or may be accomplished by techniques known to one skilled in the art like spray coating, pouring active ingredient in solution or suspension or dispersion form in a volatile solvent and allowing the solvent to evaporate leaving the active intact with the tablet in the indent(s), pouring active ingredient in a solution or suspension or dispersion in a molten material with or with out other pharmaceutically acceptable excipients and allowing the same to get solidify leaving the active along with solidified material intact within the indentation(s) and the like, such that indentation(s) are partially or completely filled with the active agent composition.

Solvents that are used in the present invention to deposit the active agent in the indent include isopropyl alcohol, ethanol, methanol, acetone, dichloromethane, ethyl acetate and other pharmaceutically acceptable solvents or mixtures thereof. Concentrations of active agent in a solvent can range from 0.1% w/v to 50% w/v.

Useful waxy materials include beeswax, carnauba wax, microcrystalline wax, polyethylene glyclols, and the like. Ratio of active agent to waxy material can range from 1:10 to 10:1.

Other pharmaceutically acceptable excipients used along with active agents in the deposit include but are not limited to binders such as gelatin, gums (acacia, tragacanth, guar gum, xanthane gum and the like), celluloses (hydroxypropyl cellulose, hydroxypropyl methylcellulose, ethylcellulose, methylcellulose, carboxy methylcellulose and the like), povidone (K30, K60, K90 and the like) or other polymeric materials or combinations thereof. Ratio of active agent to pharmaceutically acceptable excipient can range from 1:100 to 100:1.

The present invention thus provides stable compositions for reducing drug-drug or drug-excipient interactions associated with the simultaneous administration of more than one active from the same composition or provides incompatible actives in an immediate release or modified release formulation or both concomitantly from a single pharmaceutical composition.

The compositions of the present invention may be administered to humans or animals for treatment of condition(s) or disease(s) of one or more organ systems.

The following examples will further illustrate certain aspects and embodiments of the invention in greater detail and are not intended to limit the scope of the invention in any manner.

### EXAMPLE 1

### Diclofenac Sodium Tablets Deposited with Misoprostol as a Band

| | |
|---|---|
| Ingredients | mg/Tablet |

Core

| | |
|---|---|
| Diclofenac sodium | 50 |
| Lactose monohydrate | 81.25 |
| Microcrystalline cellulose | 80.63 |
| Corn starch | 52.5 |
| Povidone K 30 | 30 |
| Magnesium stearate | 5.62 |
| Water | 90 |

Band

| | |
|---|---|
| Misoprostol | 0.2 |
| Hydroxypropyl methylcellulose | 20 |
| Dichloromethane | 52.53 |

Diclofenac, lactose monohydrate, microcrystalline cellulose and corn starch were sifted through an ASTM 40 mesh sieve and mixed for 5 minutes in a rapid mixer granulator. Povidone K 30 was dissolved in water and added slowly to granulate the above dry mixture. Granules were dried using a fluid bed drier at 60° C. until the moisture content was not more than 2% when measured using an infrared moisture balance at 105° C. Dried granules were sifted through an ASTM 20 mesh sieve. Sifted granules and magnesium stearate were blended in a double cone blender for 5 minutes. Blended granules were compressed as tablets using 14.6×6 mm capsule shaped tools. Resulting tablets were banded with misoprostol, as a misoprostol-hydroxypropyl methylcellulose solution in dichloromethane, using a conventional capsule banding machine.

### EXAMPLE 2

### Enteric Coated Diclofenac Sodium Tablets Deposited with Misoprostol as a Band

| | |
|---|---|
| Ingredients | Mg/Tablet |

Core

| | |
|---|---|
| Diclofenac sodium | 50 |
| Lactose monohydrate | 81.25 |
| Microcrystalline cellulose | 80.63 |
| Corn starch | 52.5 |
| Povidone K30 | 30 |
| Magnesium stearate | 5.62 |
| Water | 90 |

Enteric coating

| | |
|---|---|
| Methacrylic acid copolymer Type C | 11.04 |
| Sodium hydroxide | 0.141 |
| Talc | 2.208 |
| Triethyl citrate | 1.08 |
| Water | 1.60 |

Band

| | |
|---|---|
| Misoprostol | 0.2 |
| Hydroxypropyl methylcellulose | 20 |
| Dichloromethane | 52.52 |

Core tablets were prepared in a manner similar to Example 1, core tablets were coated with a dispersion of methacrylic acid copolymer, sodium hydroxide, talc and triethyl citrate in water using conventional coating techniques, and the resulting coated tablets were banded with misoprostol, using a misoprostol-hydroxypropyl methylcellulose solution in dichloromethane and using a conventional capsule banding machine.

### EXAMPLE 3

### Enteric Coated Diclofenac Sodium Tablets Deposited with Misoprostol as a Band

| | |
|---|---|
| Ingredients | mg/Tablet |

Core

| | |
|---|---|
| Diclofenac sodium | 75 |
| Lactose monohydrate | 19.5 |
| Microcrystalline cellulose | 182.7 |
| Corn starch | 12.6 |
| Povidone K 30 | 7.2 |
| Magnesium stearate | 3 |
| Water | 90 |

Enteric coating

| | |
|---|---|
| ACRYL-EZE ™ | 18 |
| Water | 72 |

Band

| | |
|---|---|
| Misoprostol | 0.2 |
| Povidone K 30 | 20 |
| Dichloromethane | 52.5 |

* ACRYL-EZE ™ is a pre-formulated water-dispersible enteric coating material containing EUDRAGIT ™ L100-55 (a 1:1 copolymer of methacrylic acid and ethyl acrylate), sold by Colorcon Asia Pvt Ltd., Goa, India.

Core tablets were prepared similarly to Example 1, core tablets were coated with dispersion of ACRYL-EZE in water using conventional coating techniques and resulting coated tablets were banded with misoprostol, using misoprostol-povidone solution in dichloromethane using a conventional capsule banding machine.

### EXAMPLE 4

### Enteric Coated Diclofenac Sodium Tablets Deposited with Misoprostol as a Band

| | |
|---|---|
| Ingredients | mg/tablet |

Core

| | |
|---|---|
| Diclofenac sodium | 75 |
| Lactose monohydrate | 19.5 |
| Microcrystalline cellulose | 182.7 |
| Corn starch | 12.6 |
| Povidone K 30 | 7.2 |
| Magnesium stearate | 3 |
| Water | 90 |

Enteric coating

| | |
|---|---|
| ACRYL-EZE | 18 |
| Water | 72 |

Band

| | |
|---|---|
| Misoprostol | 0.2 |
| Hydroxypropyl methylcellulose | 20 |
| Dichloromethane | 52.52 |

Core tablets were prepared in a manner similar to Example 1, core tablets were coated with dispersion of ACRYL-EZE in water using conventional coating techniques and resulting coated tablets were banded with misoprostol, using misoprostol-hydroxypropyl methylcellulose solution in dichloromethane using conventional capsule banding machine.

### EXAMPLE 5

### Diclofenac Sodium Capsules Banded with Misoprostol

| | |
|---|---|
| Ingredients | mg/Tablet |

Capsule

| | |
|---|---|
| Diclofenac sodium | 50 |
| Lactose monohydrate | 13 |
| Microcrystalline cellulose | 12.9 |
| Magnesium stearate | 5.62 |

Band

| | |
|---|---|
| Misoprostol | 0.2 |
| Hydroxypropyl methylcellulose | 20 |
| Dichloromethane | 52.52 |

Diclofenac sodium, lactose monohydrate, microcrystalline cellulose were sifted using an ASTM 20 mesh sieve, magnesium stearate was sifted using an ASTM 40 mesh sieve and all the materials were mixed for 5 minutes using a double cone blender. Blend was filled into hard gelatin capsules using a hand operated capsule filling machine. Filled capsules were banded with misoprostol using a misoprostol and hydroxypropyl methylcellulose dispersion in dichloromethane, using a conventional capsule banding machine.

### EXAMPLE 6

### Diclofenac Sodium Tablets Printed with Misoprostol

| | |
|---|---|
| Ingredients | mg/Tablet |

Core

| | |
|---|---|
| Diclofenac sodium | 50 |
| Lactose monohydrate | 81.25 |
| Microcrystalline cellulose | 80.63 |
| Corn starch | 52.5 |
| Povidone K 30 | 30 |
| Magnesium stearate | 5.62 |
| Water | 90 |

Printing composition

| | |
|---|---|
| Misoprostol | 0.2 |
| Hydroxypropyl methylcellulose | 20 |
| Triacetin | 2 |
| Dichloromethane | 50 |

Diclofenac sodium core tablets were prepared in a manner similar to Example 1. Misoprostol printing material was prepared by dissolving misoprostol, hydroxypropyl methylcellulose and triacetin in dichloromethane. Core tablets were imprinted with the misoprostol printing composition using a high density printing machine.

### EXAMPLE 7

### Bilayer Diclofenac Sodium Tablets Having Depressions Filled with Misoprostol

| | |
|---|---|
| Ingredients | mg/Tablet |

Core (Part 1)

| | |
|---|---|
| Diclofenac sodium | 75 |
| Lactose monohydrate | 19.5 |
| Microcrystalline cellulose | 19.5 |
| (Avicel PH 101) | |
| Povidone K 30 | 7.2 |
| Corn starch | 12.6 |
| Microcrystalline cellulose | 361.2 |
| (Avicel PH 102) | |
| Magnesium stearate | 5 |
| Water | 144 |

Core (Part 2)

| | |
|---|---|
| Microcrystalline cellulose | 195 |
| (Avicel PH 102) | |
| Magnesium stearate | 5 |

Enteric coating

| | |
|---|---|
| ACRYL-EZE ™ | 70 |
| Silicon dioxide | 0.35 |
| Water | 288 |

Deposit

| | |
|---|---|
| Misoprostol | 0.2 |
| Povidone K 30 | 10 |
| Ethyl acetate | 94.85 |
| Methanol | 40.65 |

Manufacturing procedure:
1. Diclofenac sodium, lactose anhydrous, Avicel PH 101 and corn starch were passed through an ASTM 20 mesh sieve and mixed well.
2. Povidone K30 was dissolved in purified water.
3. Blend of step 1 was granulated with step 2 solution.
4. Granules were dried in fluid bed dryer at 60° C., until the moisture content of the granules was about 2% when measured using an infrared moisture balance at 105° C.
5. Granules were passed through an ASTM 20 mesh sieve.
6. Magnesium stearate was passed through an ASTM 40 mesh sieve.
7. Blend of step 5 was lubricated with step 6 material.
8. Avicel PH 102 was passed through an ASTM 20 mesh sieve.
9. Magnesium stearate was passed through an ASTM 40 mesh sieve.
10. Avicel PH 102 was combined with step 9 material.
11. Blend of step 7 was loaded in to a first hopper of a bilayer tablet compression machine.
12. Blend of step 10 was loaded in to a second hopper of the machine.
13. Bilayer tablets were compressed using 11 mm punches having cup shaped projections.

### Enteric Coating

14. ACRYL-EZE and silicon dioxide were dispersed in water to form a uniform dispersion.
15. Tablets of step 13 were coated with the coating dispersion of step 14, in a perforated coating pan.

### Misoprostol Deposit

16. Misoprostol and Povidone K30 were dissolved in a mixture of ethyl acetate and methanol.
17. Solution of step 16 was filled into cup shaped indentations of the tablets using a micropipette.
18. The resulting tablets were air dried for 2 hours at room temperature.
19. The tablets of step 3 were further dried in a tray dryer at 40° C. for 12 hours.

A stability study of composition of Example 7 was carried out for misoprostol. The product was stored at 40° C. and 75% relative humidity, and analyzed at intervals by high performance liquid chromatography for the presence of impurities. The following table depicts the level of an impurity detected by high performance liquid chromatography and denoted “A,” expressed in area-percent.

| Storage | Impurity A |
|---|---|
| Initial | 0.15 |
| 1 Month | 0.38 |
| 2 months | 0.53 |

### EXAMPLE 8

### Double Layer Diclofenac Sodium Tablets Having Depressions Filled with Misoprostol

| | |
|---|---|
| Ingredients | mg/Tablet |

Core (Part 1)

| | |
|---|---|
| Diclofenac sodium | 75 |
| Lactose monohydrate | 19.5 |
| Microcrystalline cellulose | 265.9 |
| Povidone K 30 | 16 |
| Corn starch | 12.58 |
| Sodium starch glycolate | 6 |
| Magnesium stearate | 5 |

Core (Part 2)

| | |
|---|---|
| Microcrystalline cellulose | 192 |
| Povidone K 30 | 4 |
| Sodium starch glycolate | 3 |
| Magnesium stearate | 1 |

Seal coating

| | |
|---|---|
| Hydroxypropyl methylcellulose | 12 |
| Water | 288 |

Enteric coating

| | |
|---|---|
| Eudragit L 100 55 | 33.48 |
| Triethyl citrate | 6.72 |
| Talc | 11.16 |
| Sodium hydroxide | 0.456 |
| Water | 207.26 |

Deposit

| | |
|---|---|
| Misoprostol | 0.2 |
| Povidone K 30 | 10 |
| Ethyl acetate | 6.09 |
| Methanol | 2.03 |

Manufacturing procedure:
1. Diclofenac sodium, lactose anhydrous, microcrystalline cellulose, corn starch and sodium starch glycolate were passed through an ASTM 20 mesh sieve and mixed well.
2. Povidone K30 was dissolved in water.
3. Blend of step 1 was granulated with step 2 solution.
4. Granules were dried in a fluid bed dryer at 60° C. until the moisture content of the granules was about 2% when measured using an infrared moisture balance at 105° C.
5. Granules were passed through an ASTM 25 mesh sieve.
6. Magnesium stearate was passed through an ASTM 40 mesh sieve.
7. Blend of step 5 was lubricated with step 6 material.
8. Microcrystalline cellulose, povidone and sodium starch glycolate were sifted through an ASTM 40 mesh sieve and mixed in a double cone blender for 5 minutes, then lubricated with magnesium stearate in a double cone blender for 2 minutes.
9. Blend of step 7 was loaded into a first hopper of a bilayer tablet compression machine.
10. Blend of step 8 was loaded into a second hopper of the machine.
11. Bilayer tablets were compressed using 12 mm punches having cup shaped projections.

### Enteric Coating

12. Eudragit was dispersed in purified water to form uniform dispersion.
13. Dispersion of step 12 was neutralized with 1% w/v sodium hydroxide solution in water.
14. Triethyl citrate was added and stirred for 30 minutes.
15. Bilayer tablets of step 11 were coated the using dispersion of step 14.

### Misoprostol Deposit

16. Misoprostol and Povidone K30 were dissolved in a mixture of ethyl acetate and methanol.
17. Solution of step 16 was filled into cup shaped indentations of the tablets using a micropipette.
18. The resulting tablets were air dried for 2 hours at room temperature.
19. The tablets of step 3 were further dried in a tray dryer at 40° C. for 12 hours.

### EXAMPLE 9

### In-Vitro Dissolution Profile of the Product of Example 8

Apparatus: rotating paddle (USP Type II)

### Stirring rpm: 50

Media: 0.1 N HCl for 2 hours, followed by pH 6.8 phosphate buffer

| Medium | Time (minutes) | % Drug Release |
|---|---|---|
| 0.1 N HCl | 120 | 0 |
| pH 6.8 Phosphate | 10 | 9 |
| buffer | 20 | 29 |
| | 30 | 49 |
| | 45 | 72 |
| | 60 | 81 |
| | 90 | 86 |

1. A solid pharmaceutical dosage form, comprising a substrate containing an active agent and a solid composition deposited onto areas of a surface of the substrate and containing an active agent.

2. The solid pharmaceutical dosage form of claim 1, wherein the substrate and the solid composition contain the same active agent.

3. The solid pharmaceutical dosage form of claim 1, wherein the solid composition releases active agent at a rate different from the release rate from the substrate.

4. The solid pharmaceutical dosage form of claim 1, wherein the active agent in the solid composition is different from the active agent in the substrate.

5. The solid pharmaceutical dosage form of claim 1, wherein the active agent in the solid composition is chemically incompatible with the active agent in the substrate.

6. The solid pharmaceutical dosage form of claim 1, wherein the substrate comprises more than one layer, each layer containing the same or different active agents.

7. The solid pharmaceutical dosage form of claim 1, wherein the solid composition is deposited in a fluid form, and becomes solid.

8. The solid pharmaceutical dosage form of claim 1, wherein the solid composition is deposited into one or more recessed areas on at least one surface of the substrate.

9. The solid pharmaceutical dosage form of claim 1, wherein the solid composition is applied as a band onto the substrate.

10. The solid pharmaceutical dosage form of claim 1, wherein the solid composition is applied as an imprint onto the substrate.

11. A solid pharmaceutical dosage form, comprising a substrate containing an active agent and a solid composition deposited into one or more recessed areas on at least one surface of the substrate and containing a different active agent.

12. The solid pharmaceutical dosage form of claim 11, wherein the active agent in the solid composition is chemically incompatible with the active agent in the substrate.

13. The solid pharmaceutical dosage form of claim 11, wherein the substrate comprises more than one layer, each layer containing the same or different active agents.

14. The solid pharmaceutical dosage form of claim 11, wherein the solid composition is deposited in a fluid form, and becomes solid.

15. The solid pharmaceutical dosage form of claim 11, wherein a substrate has more than one solid composition deposited into recessed areas, each solid composition containing at least one active agent different from an active agent present in the substrate.

16. The solid pharmaceutical dosage form of claim 11, wherein the substrate comprises a first layer containing an active agent, a second layer containing no active agent, and a solid composition deposited onto a surface of the second layer.

17. A solid pharmaceutical dosage form, comprising a substrate containing an active agent and a solid composition deposited as a band onto at least one surface of the substrate and containing a different active agent.

18. The solid pharmaceutical dosage form of claim 17, wherein the substrate is a capsule.

19. The solid pharmaceutical dosage form of claim 17, wherein the solid composition is deposited in a fluid form, and becomes solid.

20. The solid pharmaceutical dosage form of claim 17, wherein the active agent in the solid composition is chemically incompatible with the active agent in the substrate.
